(19) United States
(12) Patent Application Publication
(10) Pub. No.: US 2007/0141067 A1
(43) Pub. Date: Jun. 21, 2007

(54) TARGETED CANCER THERAPY

(75) Inventor: Svetomir N. Markovic, Rochester, MN (US)

Correspondence Address: FISH & RICHARDSON P.C., PO BOX 1022, MINNEAPOLIS, MN, 55440-1022, US

(73) Assignee: MAYO MEDICAL VENTURES, Rochester, MN (US)

(21) Appl. No.: 11/670,326

(22) Filed: Feb. 1, 2007

Related U.S. Application Data
(62) Division of application No. 10/779,159, filed on Feb. 13, 2004.

(51) Int. Cl. A61K 48/00; C12Q 1/68; G06F 19/00; A61K 39/395

(52) U.S. Cl. 424/155.1; 435/6; 702/20; 514/44

(57) ABSTRACT

Methods and compositions are described for determining the expression profile of a tumor and subsequently determining an appropriate cancer therapy. Accordingly, a database can correlate expression profile data of a particular tumor before a chemotherapeutic is administered, and after the chemotherapeutic is administered and after tumor progression, such as when a tumor has developed a resistance to the chemotherapeutic agent. The database can then be used to determine an appropriate treatment for a patient with a particular kind of tumor that expresses a particular subset of genes. The methods and compositions related to the invention can further be used to predict at least one secondary therapeutic agent, which is targeted against a gene overexpressed in tumor tissue following treatment with a primary therapy and during tumor progression.

### CROSS-REFERENCE TO RELATED APPLICATIONS

This application is a divisional application of and claims priority to U.S. application Ser. No. 10/779,159, filed on Feb. 13, 2004.

### TECHNICAL FIELD

This invention relates to the field of personalized medicine, and more particularly to methods of determining cancer treatments based on gene expression profiles and response to therapy.

### BACKGROUND

The advent of the human genome project has provided advances in cell biology and the development of targeted drug therapy. For example, targeted therapies can be directed towards specific and unique features of tumor cells. The development of drug-tumor specificity has provided cancer treatment options that are safer and more effective than systemic therapies that induce maximally tolerated toxicity. Gleevac, for example, is a drug directed at the gene product of c-kit in chronic myelogenous leukemia. Gleevac is active against the same target molecule in gastrointestinal stromal tumors (Joensuu et al., N. Engl. J. Med. 344:1052-6, 2001). Thus, two seemingly different malignancies share a “genetic abnormality” that allows them to respond to therapy with the same drug. Similar observations have been reported with the use of Herceptin for the treatment of metastatic salivary gland tumors expressing up-regulation of her-2/neu (Haddad et al., Oral Oncol. 39:724-7, 2003).

### SUMMARY

The new methods and compositions featured in the invention are related to methods of determining an appropriate cancer therapy by assessing the expression profile of a tumor. Accordingly, a database can be established that correlates expression profile data of a particular tumor before a chemotherapeutic is administered, and after the chemotherapeutic is administered and after tumor progression following the administration, such as when a tumor develops a resistance to the chemotherapeutic agent. The database can include information relating to the tumor (type, size, stage, etc.), and the response of the tumor and the patient to treatment. This information can be associated with the particular phenotype of the tumor. The database can then be used to determine an appropriate treatment for a patient with a particular kind of tumor that expresses a particular subset of genes. The methods described herein place primary importance on the genetic profile (i.e., gene expression pattern) of a tumor for determination of the appropriate treatment. The methods and compositions related to the invention can further be used to predict at least one secondary therapeutic agent to complement the therapeutic activity of the primary therapy. Accordingly, the secondary therapeutic agent is targeted against a gene that is found to be overexpressed or underexpressed in tumor tissue following treatment with a primary therapy and further tumor progression.

Accordingly, a computer-accessible medium that includes a database is a feature of the invention. The database includes a plurality of records that associates tumor identification information (e.g., tumor type, size, etc.) with the gene expression pattern of the tumor prior to treatment with a primary cancer therapy. A primary therapy can be a radiation therapy, chemotherapy, or another like therapy. Each record of a database can also include gene expression data from the tumor after treatment with a primary therapy and following tumor progression. Furthermore, information relating to the effect of the primary therapy on tumor progression can be included in the database. In addition to gene expression data, the database can record gene amplification or deletion data from the tumor following treatment with the primary therapeutic. Gene expression data and genomic amplification data can be obtained by nucleic acid array technology, including but not limited to the use of microarrays. Gene amplification or deletion information can be determined by comparative genomic hybridization (CGH).

Also described herein, as a feature of the invention, is an article of computer-readable medium with encoded instructions (e.g., a software program). The encoded instructions can effect the processing of information regarding the gene expression profile of a tumor prior to treatment with a cancer therapy to suggest an appropriate primary therapy to treat the tumor. Optionally, the program can suggest an appropriate dosage and treatment regimen. The program can further receive information regarding a gene expression profile of the tumor following treatment with a primary therapy and can further suggest at least one secondary agent, for use in a combination therapy. The program can make a suggestion based on a collection of data (such as in the form of a database) that correlates gene expression information before treatment of a tumor with a particular chemotherapy with gene expression information following treatment with a particular chemotherapy. The secondary agent will, for example, target a gene that is typically modified (upregulated or downregulated) following treatment with the chemotherapeutic agent and continues to be modified during tumor progression. By “targeting” the gene, the secondary agent can alter its activity. For example, if a gene is upregulated during tumor progression, the secondary therapeutic agent can decrease its activity, or decrease the activity of an RNA or protein expressed from the gene. The secondary agent can alternatively target a gene that is typically downregulated following the chemotherapy. Such a secondary agent will, for example, stimulate transcription or otherwise compensate for the decrease in gene activity. The secondary therapeutic agent can be, for example, an siRNA, antisense RNA, antibody or small molecule drug. The program can present its treatment suggestions in print or in an electronic format. A software program is an article of computer-readable medium having instructions encoded thereon. The instructions enable the program to process the given information and subsequently deliver an appropriate treatment suggestion.

Other features of the invention are new methods of treating a patient having a tumor. According to one exemplary method, the gene expression profile of the tumor is determined and compared with information in a database as described above. A primary therapy to treat the tumor is then selected based on the information from the database. A secondary therapeutic agent can also be administered to alter the activity of a gene identified as being modified (upregulated or downregulated) (or predicted to be modified based on information from the database) following administration of the primary therapy and during tumor progression. In some methods, an expression profile will be determined following treatment with the primary therapy, and prior to treatment with the secondary therapy. The secondary therapy can be, for example, an siRNA, antisense RNA, antibody, or small molecule inhibitor.

Methods of selecting a drug profile for a cancer patient (e.g., a patient having a tumor) are also the invention. According to one exemplary method, an expression profile of a tumor is determined, and the expression profile is compared to information in a database, such as a database described herein. An appropriate chemotherapeutic drug is selected based on the information in the database.

Also described herein are methods for identifying a therapeutic agent to treat a tumor. By one exemplary method, a test sample and a control sample of tumor cells are provided. The test sample is contacted with a primary anti-tumor drug and a test secondary therapeutic agent. The control sample is only contacted with the primary drug. The test sample and control sample are then assayed for survivability. A decrease in cell survivability in the test sample as compared to the control sample is an indication that the test secondary therapeutic agent can be used to treat a tumor.

Other features of the invention include nucleic acid arrays and methods of generating nucleic acid arrays. One exemplary method includes attaching a set, or subset, of capture probes (or cDNAs) to a substrate, and the set can represent a subset of the complete genome. A set of capture probes can include sequences complementary to RNAs expressed in a tumor before administration of an anti-tumor therapy, as well as sequences complementary to RNAs expressed in a tumor after administration of an anti-tumor therapy and after tumor progression. The set of capture probes can also be complementary to a subset of all the RNAs that are expressed in a tumor before administration of an anti-tumor therapy and after administration of an anti-tumor therapy and after tumor progression. Subsets of RNAs expressed in a tumor before and after administration of the therapy can be assayed from a single patient. The subset of capture probes attached to the array can be tumor specific, such as for monitoring expression of genes in a metastatic malignant melanoma. The subset can also be tumor stage specific.

Methods for monitoring a tumor in a patient are also provided. One exemplary method includes determining a gene expression profile of a tumor before administration of an anti-tumor therapy and again after administration of the therapy. Comparison of the gene expression profiles can allow for the identification of a gene that has modified expression in response to the anti-tumor therapy.

Other methods of treating a patient having a tumor include determining a gene expression profile of the tumor; administering a primary therapy to treat the tumor, determining a gene expression profile of the tumor after therapy, and comparing the profiles from before and after therapy to identify a gene that is modified (upregulated or downregulated) following administration of the anti-tumor therapy. Depending on what gene or genes are identified, a secondary therapeutic agent can be selected based on its ability to alter activity of the gene, or an RNA or protein expressed from the gene. Alternatively, a secondary therapeutic agent can be selected based on its ability to stimulate gene expression or protein activity from a downregulated gene.

An anti-tumor therapy, as referred to herein, can be any therapy for the purpose of decreasing or eliminating a tumor. The therapy can be a chemotherapy, such as a drug, or a radiation therapy. The therapy can include a second therapeutic agent, such as a gene-specific (or non-gene-specific) therapeutic agent, such as an siRNA, antisense RNA, triple helix RNA, ribozyme, antibody, or small molecule inhibitor.

The methods and compositions related to the invention can be used to achieve enhanced anti-tumor efficacy because treatment is selected based on the genotype of the tumor, instead of, or in addition to the appearance of a tumor. Because the therapy is targeted to the specific tumor, the methods may also be accompanied by fewer side effects. In addition, a patient will not be administered a drug or therapeutic agent if it is determined that the occurrence of a particular molecular phenotype indicates that they are unlikely to benefit from the treatment.

Unless otherwise defined, all technical and scientific terms used herein have the same meaning as commonly understood by one of ordinary skill in the art to which this invention pertains. Useful methods and materials are described below, although similar or equivalent methods and materials can be used in the practice or testing of the present invention. The materials, methods, and examples are illustrative only and not intended to be limiting. Other features and advantages of the invention will be apparent from the accompanying drawings and description, and from the claims. The contents of all references, pending patent applications and published patents cited throughout this application are hereby expressly incorporated by reference. In case of conflict, the present specification, including definitions, will control.

### DESCRIPTION OF DRAWINGS

FIG. 1A is a graph plotting the log₂ fluorescence ratios of chromosome 18 oligonucleotide array probes (y-axis) as a function of chromosomal position (x-axis). The numbers along the top of the graph represent the position (in MB) along chromosome 18. Arrows indicate the cytogenetically defined breakpoint region in each cell line (16447, 50122, and 16455 cell lines).

FIG. 1B is a graph plotting the log₂ fluorescence ratios of X chromosome oligonucleotide array probes (y-axis) as a function of chromosomal position (x-axis). The numbers along the top of the graph represent the position (in MB) along the X chromosome. The data was collected from five X-series cell lines containing the indicated number of X chromosomes.

FIG. 2A is a photomicrograph of cells from a pancreas cancer cell line (Panc1) treated with Lamin B1 siRNA (see Example 3). Cells were fixed and stained with rabbit anti-vimentin antibody. Secondary antibodies used were FITC-goat anti-rabbit antibodies.

FIG. 2B is a photomicrograph of cells from a pancreas cancer cell line (Panc1) treated with Lamin B1 siRNA (see Example 3). Cells were fixed and stained with mouse anti-Lamin B1 antibody. Secondary antibodies used were rhodamine goat anti-mouse.

FIG. 2C is a photomicrograph of cells from a pancreas cancer cell line (Panc1) treated with Lamin B1 siRNA (see Example 3). Cells were fixed and stained with DAPI to visualize cellular DNA.

FIG. 3 is a graph illustrating transfection efficiencies of 13 different transfection reagents. Black vertical bars represent percent viability, and gray bars represent percent decrease in GFP expression. Efficiency of silencing was calculated by adding the percent viability and the percent of GFP silencing (see Example 3).

FIG. 4 is a graph demonstrating the results of a high throughput RNAi functional validation screen of 139 cancer genes (278 different siRNAs) for effects on HeLa cell survival (see Example 4). Cell viability was determined using Cell Titer Blue Reagent (Promega). The y-axis represents relative fluorescent units (RFU) calculated from 4 replicate transfection experiments.

FIG. 5 is a graph summarizing the percent cell survival resulting from treatment with 42 different siRNAs (see FIG. 4), all of which caused a significant decrease in cell survival (“Hit CutOff at 30,000 RFU” (black bars) plus “Cutoff at 23,600 RFU: 50% level” (gray bars)). Using the 50% cutoff (“Cutoff at 23,600 RFU: 50% level” (gray bars)) reduced the number of positives to only 5%. The average negative control (“Avg. −ve Control,” last bar on the right) was calculated by averaging all of the control treatments described in FIG. 3. The error bars in all cases indicate the standard deviation of four separate siRNA treatments.

FIG. 6A is a graph illustrating percent cell survival following treatment with a gene-specific siRNA, with or without a low dose drug. Seventy-six test siRNAs (2 or 3 siRNAs per gene plus 6 control siRNAs), targeting 29 novel candidate genes, were transfected into HeLa cells. The graph shows cell survival following siRNA pretreatments without drug (gray bars) and with 0.5 μg/ml doxorubicin (black bars).

FIG. 6B is an enlarged section of the graph in FIG. 6A. The figure shows that siRNA C has a much greater effect on cell viability in the presence of low dose doxorubicin (circled data).

FIG. 6C is a graph of the same data of FIG. 6A plotted as a percentage increase in sensitivity relative to the untreated sample.

### DETAILED DESCRIPTION

The new methods and compositions described herein are related to methods of determining an appropriate cancer therapy by assessing the gene expression profile of a tumor. Accordingly, the tumor of a patient is characterized by array technology before a chemotherapeutic is administered, and after the chemotherapeutic is administered to determine a “molecular phenotype” of the tumor. Comparison of gene expression patterns before and after treatment will reveal at least (i) a cell population(s) having a gene expression pattern that is eliminated following treatment (a “sensitive molecular phenotype”), (ii) a cell population(s) exhibiting a new gene expression pattern following treatment (an “acquired molecular phenotype”), and (iii) a cell population(s) having a gene expression pattern that is static following treatment (a “persistent molecular phenotype”). The acquired and persistent molecular phenotypes are collectively called “resistant molecular phenotypes.” The gene expression data can be correlated with phenotypic parameters, including clinical outcome (such as survival data, tumor growth or remission, and the like), and demographic data (such as gender, age, weight, etc.). From this data, correlations can be drawn that can predict the clinical outcome resulting from treatment with a particular kind of chemotherapeutic. A database can be generated from this information, and the database can be used to predict the most appropriate primary therapeutic to treat a particular tumor in a particular patient. The methods and compositions related to the invention can further be used to predict at least one secondary therapeutic agent, to complement the therapeutic activity of the primary therapy. Typically, the secondary therapeutic agent is targeted against a gene or genes whose expression is characterized as contributing to or creating a resistant molecular phenotype.

Nucleic acid arrays can be used to generate gene expression data of tumor cells. The arrays can be used to generate data before treatment with a primary drug or therapy. The primary drug can be a chemotherapeutic agent or a radiation therapy, for example. Gene expression data are also collected following treatment with the primary drug and after tumor progression. Tumor progression is marked by an increase in the size of the tumor or a regrowth of the tumor following a period of remission and/or a period when the tumor was diminished in size. Tumor progression is an indication that the tumor is no longer (or is not ever) responding to treatment with the primary drug. In other words, the tumor has lost a degree of sensitivity to the drug or has developed a degree of resistance to the drug. Genetic profiling of the tumor, such as by a nucleic acid array technology, before treatment begins and after the tumor has developed a resistance to the primary therapy, provides information about genes that may be contributing to the resistance.

A gene that exhibits a change in expression levels (e.g., an increase or a decrease in expression) following tumor progression and therapy may be a gene important for maintaining the sensitivity of the tumor to the primary therapy. Alternatively, or in addition, the gene may be important for the resistance that the tumor acquires against the primary drug. Therefore, the gene can be a target of a secondary therapeutic agent, which will function to decrease the activity of the gene if it is upregulated, or increase the activity (or otherwise compensate for the activity) of the gene if it is downregulated. The use of the secondary therapy can thereby increase or prolong the tumor's sensitivity to the primary drug.

As described herein the data generated by nucleic acid arrays can be used to design combination therapies to treat specific tumor types. A combination therapy can include one secondary therapeutic agent that will target a specific gene discovered to have a resistant molecular phenotype in response to a primary therapy, or the combination therapy can include more than one secondary therapeutic agent, each of which can target an individual gene product that was found to have a resistant molecular phenotype.

The secondary therapeutic agent can target a gene that is represented in a resistant molecular phenotype in response to a primary therapy. Thus the secondary therapeutic agent can target a gene that is upregulated or down regulated in response to the primary therapy and following tumor progression. For example, the secondary therapeutic agent can target an RNA or protein encoded by an upregulated gene. The secondary therapeutic agent can be, for example, a ribozyme, triple-helix molecule, siRNA or antisense RNA to target the overexpressed RNA; or the agent can be, for example, an antibody or small molecule inhibitor to target the overexpressed protein. In another example, the secondary therapeutic agent can target a downregulated gene. The secondary therapeutic agent can be an RNA, protein or small molecule that stimulates transcription of the downregulated gene, increases stability of RNA transcribed from the gene, modulates splicing of the RNA transcribed from the gene, or otherwise increases gene activity.

The array-based methods of phenotyping tumor cells before and after treatment can be applied to a variety of tumors, including, but not limited to, melanomas (e.g., metastatic malignant melanomas), sarcomas (e.g., lymphosarcomas), gliomas, carcinomas (e.g., choriocarcinomas and bronchogenic carcinoma), myelomas (e.g., multiple myelomas), neuroblastomas, leukemias, and cancers of the lung, breast, colon, prostate, skin, ovaries, and bladder. The array-based assays featured in the invention can be performed at multiple stages of tumor progression, such as throughout the survival time of the patient.

The array-based methods can be used to catalogue a response of a tumor cell to a particular cancer therapy, such as a chemotherapeutic or radiation therapy. Exemplary chemotherapeutics include, but are not limited to, cisplatin, dacarbazine, carmustine, interferon-α, interleukin-2, temozolomide, paclitaxel, capecitibine, cladribine, fludarabine, methotrexate, bleomycin, etoposide, chlorambucil, thiotepa, and busulfan.

In addition to gene expression analysis, comparative genomic hybridization (CGH) can be performed to monitor changes at the genomic level in response to tumor progression, and in response to a therapy (e.g., a chemotherapeutic). For example, CGH can detect gene duplications or genetic deletions. CGH can further reveal hemizygous or homozygous deletions in the germline or in a cancer cell.

To perform the array-based methods featured in the invention, nucleic acid (e.g., DNA or RNA) is isolated from a tissue, such as a tissue from a biopsy, or a scraping, or a surgical procedure. The nucleic acid is labeled, such as with a fluorescent dye (e.g., Cy3 or Cy5), and the labeled nucleic acid is applied to a microarray or gene chip. For example, for CGH, genomic DNA can be isolated from tumor tissue and from normal tissue, the genomic DNA labeled with unique fluorescent labels by a method such as random priming. One label, such as Cy3, can be used to label DNA isolated from normal tissue; and a second label, such as Cy5, can be used to label DNA isolated from cancer tissue. The chromosome integrity from each tissue can be compared by array analysis. CGH can be used at single gene resolution to determine gene copy number before and after tumor progression. In another example, such as for gene expression analysis, RNA can be isolated from tumor tissue before initiation of treatment with a primary therapy, and after treatment and tumor progression. The RNA can be labeled with unique fluorescent labels by a method such as nucleotide incorporation during PCR (e.g., with Cy3-dUTP or Cy5-dUTP), or by the use of labeled primers for reverse transcription and/or PCR. One label, such as Cy3, can be used to label RNA isolated before cancer treatment; and a second label, such as Cy5, can be used to label RNA isolated after treatment and tumor progression. Gene expression in each tissue can be compared by array analysis. Additional array-based methods are described below.

Genes that are identified as having copy number changes (such as by CGH technology) and as being over- or underexpressed (such as by expression array technology) can be determined to be targets for tumor specific secondary therapies. It is not essential that a gene satisfy both criteria (i.e., have a change in copy number and a corresponding change in expression levels) to be a target for a secondary therapeutic; a gene will preferably meet at least one of the criteria (for example, (a) the gene is overexpressed, or (b) the gene is amplified.)

Database Generation

A collection of tumor samples can provide information that contributes to a database that correlates the genotype of a cell before and after a primary therapy (e.g., radiation or chemotherapy) with clinical data that can include, but is not limited to, tumor size, survival, overall response to therapy, and demographic data such as gender, age, weight, vital statistics, etc. Nucleic acid (DNA and/or RNA) can be harvested from tissue collected by a biopsy, such as a needle biopsy, or by a tissue scraping, such as from a skin cancer (e.g., a melanoma or a basal cell carcinoma), or by surgical removal of at least a portion of a tumor. Tissue is collected prior to initiation of therapy and can also be collected at the time of tumor progression regardless of the duration of therapy. DNA, RNA can be isolated from the tissue and labeled for analysis by array technology, such as array analysis or gene chip analysis. Optionally, the tissue can be frozen and stored for a period of time (such as for a day, a week, a month, or a year, or any fraction thereof) before isolation of nucleic acid. DNA or RNA isolation from a tissue sample can be accomplished by methods known in the art (see, for example, Sambrook et al., Molecular Cloning: A Laboratory Manual, 2nd ed., Cold Spring Harbor Laboratory, Cold Spring Harbor Laboratory Press, Cold Spring Harbor, N.Y., 1989).

Microarray analysis can be performed by mixing two test samples of labeled nucleic acid and applying them to the same array for comparative analysis (Schena et al., Science 270:467-70, 1995). The array can be any array described herein, or any other array that is functional in the described analysis. For example, an RNA sample isolated from a tumor tissue before the start of therapy can be labeled and mixed with RNA isolated from a tumor tissue after tumor progression and after the initiation of therapy (e.g., chemotherapy). The nucleic acid can be labeled with a fluorescent dye, such as Cy3 or Cy5. Preferably the nucleic acid samples from each tissue are labeled with a different and distinguishable dye. For example, RNA isolated from a tumor before administration of a chemotherapeutic can be labeled with Cy3, such as in the form of Cy3-dUTP (e.g., via a PCR reaction following reverse transcription), and RNA isolated from a tumor following tumor progression and administration of a primary cancer therapy can be labeled with Cy5, such as in the form of Cy5-dUTP. For CGH analysis, genomic DNA isolated from a tumor before administration of a primary cancer therapy, and DNA isolated following tumor progression and administration of a cancer therapy can be labeled with Cy3 and Cy5, respectively (or vice versa), by a random priming method using Cy3-dUTP and Cy5-dUTP.

Tissue samples can be collected from a patient at the time of tumor progression, regardless of whether the patient has a short-lived response to therapy, a prolonged remission, or no response to therapy whatsoever. Furthermore, any cancer therapeutic is a valid test candidate for the methods described herein. For example, sensitive and resistant phenotypes can be determined following treatment of a tumor with a chemotherapeutic agent, including, but not limited to, cisplatin, dacarbazine, carmustine, interferon-α, interleukin-2, temozolomide, paclitaxel, capecitibine, cladribine, fludarabine, methotrexate, bleomycin, etoposide, chlorambucil, thiotepa, and busulfan.

Labeled nucleic acids are hybridized to an array following labeling, and unbound nucleic acids are washed away. The bound, labeled nucleic acids are detected using an appropriate method. For example, to detect fluorescence intensity at each spot on an array, a laser confocal scanner or CCD-based scanner can be used. To detect spots hybridized with radioactively labeled nucleic acids, a phosphorimager can be used.

CGH and gene expression data can be processed to prioritize gene candidates as targets for a secondary therapy. The CGH copy number data can be ordered according to the location of the clones along chromosomes. A model of the variance of the detector's response can be generated from a series of array hybridizations of normal haploid DNA vs. normal haploid DNA. Significant copy increase at individual genes can be determined based on comparisons of the values (e.g., log ratio values) of data measurements including the control data measurements. Significant differences in the distributions can be determined using statistical methods, such as the Student t distribution, or non-parametric tests, such as Kolmogorov-Smirnov statistics or TnoM (a ranks based test).

In one example, a statistical analysis of CGH data can be accomplished by the following methods. Application of the t-test will provide a statistic, S (which is assigned “+” for amplifications and “−” for deletions), for the two normal distribution fits of CGH values representing essentially the haploid and non-haploid distributions of values for that gene. This can be used to produce a score that equals the number of standard deviations by which a given gene's S score deviates from the mean of the S scores for all of the genes in the data. Amplicons can be designated by assigning a score to each genomic interval that measures how consistently amplified it appears to be. This can be calculated as Z(region)=sum(S)/sqrt(# of genes in the interval) where the sum is taken over all the genes in the interval, and the S scores are signed. Regions with a Z score that passes a user-defined threshold are reported as potentially aberrant. The influence of gene copy number on gene expression level will be evaluated as described (Hyman et al., Cancer Res. 62:6240-5, 2002). For example, same-slide normalized CGH and cDNA ratios from each cell line can be log-transformed and median-centered. cDNA data can be median-centered using values across all cell lines tested. For each gene, the CGH data can be represented by a vector that is labeled “1” for an S value above a user defined threshold and “0” for no amplification. Amplification can then be correlated with gene expression using the signal-to-noise statistics (Hyman et al., Cancer Res. 62:6240-5, 2002).

A weight, w_{g}, can be calculated for each gene:

$${w}_{g} = \frac{{m}_{g 1} - {m}_{g 0}}{{σ}_{g 1} + {σ}_{g 0}} ,$$

where (m_{g1}, and σ_{g1}) and (m_{g0} and σ_{g0}) denote the means and standard deviations for the expression levels for amplified and non-amplified cell lines, respectively. To assess the statistical significance of each weight, 10,000 random permutations of the label vector can be performed. The probability that a gene has a larger or equal weight by random permutation than the original weight can be denoted by α. A low α(<0.05) indicates a strong association between gene expression and amplification.

Genes found to have increased expression following drug administration and tumor progression, can be categorized as genes whose expression is involved in decreasing the sensitivity tumor cells to the drug. These genes can therefore be the targets of secondary therapeutic agents, such as RNAi, antisense, or ribozyme therapeutic agents, directed against the upregulated gene. The resulting downregulation of gene expression can increase the sensitivity of the tumor cells to the drug.

Information from tumor tissue arrays (see below) can also be added to a database described herein as a supplement to the gene expression data. Protein expression and genomic information from tumor tissue arrays can be incorporated into algorithms that will predict appropriate primary and secondary therapeutic agents based on tumor type and molecular phenotype.

The status of gene amplification and expression of prioritized, functionally relevant targets can be correlated to clinical parameters such as survival and response to therapy.

The data generated by the methods featured in the invention can be stored in a database, such as a computer-accessible medium. The database can be a storehouse for the information pertaining to each tumor type and its molecular phenotype (sensitive or resistant (acquired or persistent)) resulting from treatment from any and each primary therapy (e.g., radiation therapy and individual chemotherapeutic agents). The database can further store personal information, including demographic data (e.g., weight, gender, age, etc.). The database can generate information regarding the best gene targets for secondary therapeutic agents. This information can be generated based on any one or a combination of tumor type, tumor molecular phenotype, primary therapy, demographic data, and the like. The database can be continually updated with new information from newly harvested tissue samples.

A database featured in the invention can be linked to a software program that will generate a recommendation for one or both of a primary therapeutic and a secondary therapeutic, based on the information stored in the database. The software program can also recommend dosage regimens. The information generated by the software can be displayed in print or in a computer readable format. The information can also be provided in an internet-based format, allowing access to information from remote locations. Optionally, the information can be password protected so that only authorized persons can access the information.

Nucleic Acid Arrays

A nucleic acid array is a substrate, such as a glass, wafer (e.g., a silica wafer) or membrane, to which is tethered a designated set of nucleic acid molecules, called capture probes, each representing a specified gene or nucleic acid sequence. Placement of the nucleic acid probes onto the substrate can be accomplished by methods known in the art. For example, a drop (e.g., spray) method, or other mechanical method, such as the directed-flow method described in U.S. Pat. No. 5,384,261, or the pin-based method described in U.S. Pat. No. 5,288,514.

A nucleic acid array can contain a set of probes that represents the entire genome of an organism, such as a mouse or human, or an array can contain a subset of gene-specific probes. For example, the subset can include a group of genes whose expression has been determined to be modulated in response to a therapy (e.g., a chemotherapy), such as in pilot experiments, or as reported in the literature. The subset of probes can also represent genes determined to be amplified or deleted, such as by CGH, such as in pilot experiments, or as reported in the literature. Arrays that contain a subset of gene-specific probes can be designed and used to monitor gene expression or gene-copy modification in tumors of particular tissue types. For example, an array specific for use in assaying the genotype and response to a chemotherapeutic of a breast cancer tumor, can include probes that hybridize to RNAs (or cDNAs) that have been found to be over- or underexpressed in breast cancer tumors. Tumor specific arrays can be designed to specifically monitor gene expression in various tissue types, including but not limited to tumors of the colon, pancreas, ovary, and lung. Tumor specific arrays can alternatively, or in addition, include gene-specific probes that hybridize to nucleic acids observed to be over- or underexpressed in a particular tumor type, such as a melanoma, carcinoma, or glioma.

An array can include probes that will serve as controls, including positive control probes and negative control probes. A positive control probe can include a housekeeping gene, such as an RNA polymerase gene, the beta actin gene, the glyceraldehyde-3-phosphate dehydrogenase gene, the hypoxanthine phosphoribosyl-transferase 1 gene, the ribosomal protein L13a, the TATA binding protein gene, and/or the ubiquitin C gene. The nucleic acid sequences of these genes are known in the art. A synthetic positive control will hybridize to a control nucleic acid that is added to the test sample from the tumor before hybridization to the array. The synthetic positive control probe should have a sequence that is not substantially identical to any of the genes of the tissue sample being assayed, such that the labeled nucleic acid from the test sample will not hybridize to the control probe. A negative control probe should have a sequence that is not substantially identical to any of the genes of the tissue sample being assayed or to the positive control sequence. Other optional control probes include a polyA, polyT, polyG, and polyC probe, useful for measuring the effects of non-specific hybridization.

A gene array can contain tens, hundreds, or thousands of individual probes immobilized at discrete, predetermined locations (addresses or “spots”) on a solid, planar support, such as a glass, metal, or nylon support. An array can be a macroarray or microarray, the difference being in the size of the spots. Macroarrays contain spots of about 300 microns in diameter or larger and can be imaged using gel or blot scanners. Microarrays contain spots less than about 300 microns, typically less than about 200 microns, in diameter. The array can have a density of at least about 10, 50, 100, 200, 500, 1,000, 2,000, or 10,000 or more probes/cm², and ranges between. The capture probes can be single stranded, or the probes can have a structure comprising a double stranded portion and a single stranded portion.

To generate data from an array, a population of labeled cDNA representing total mRNA from a sample of a tissue of interest, such as a tumor sample is contacted with the DNA array under suitable hybridization conditions. Hybridization of cDNAs with sequences in the array is detected, such as by fluorescence at particular addresses on the solid support. Thus, a pattern of fluorescence representing a gene expression pattern in the tumor sample of a particular subject or group of subjects is obtained, for example, before administration of a therapeutic agent, and after administration of the therapeutic, after tumor progression. These patterns of gene expression can be digitized and stored electronically, such as in a digital database, for computerized analysis and comparison.

By some methods, cDNAs can be used as capture probes to form the array. Suitable cDNAs can be obtained by conventional polymerase chain reaction (PCR) techniques, such as reverse transcription coupled to PCR (RT-PCR). The length of the cDNAs can be from about 20 to 2,000 nucleotides, e.g., from about 100 to 1,000 nucleotides. Other methods known in the art for producing cDNAs can be used. The cDNA probes can be attached to a suitable solid substrate, such as a coated glass microscope slide, at specific, predetermined locations in a two-dimensional grid. For example, the substrate can be coated with polylysine, which will facilitate attachment of the cDNA. A small volume (e.g., about 5 nanoliters) of a concentrated DNA solution can be placed in each spot. Spotting can be carried out using a commercial microspotting device (sometimes called an arraying machine or gridding robot) according to the vendor's instructions. Commercial vendors of solid supports and equipment for producing DNA arrays include BioRobotics Ltd., Cambridge, UK; Corning Science Products Division, Acton, Mass.; GENPAK Inc., Stony Brook, N.Y.; SciMatrix, Inc., Durham, N.C.; and TeleChem International, Sunnyvale, Calif.

The cDNAs can be attached to the solid support by any suitable method. In general, the linkage is covalent. Suitable methods of covalently linking DNA molecules to the solid support include amino cross-linking and UV crosslinking. For guidance concerning construction of cDNA arrays according to the invention, see, e.g., DeRisi et al. (Nature Genetics 14:457-460, 1996), Khan et al. (Electrophoresis 20:223-229, 1999), Lockhart et al. (Nature Biotechnol. 14:1675-1680, 1996).

In an alternative method, immobilized DNA probes of an array are synthetic oligonucleotides. Preformed oligonucleotides can be spotted to form a DNA array, using techniques described herein with regard to cDNAs. In yet another alternative, the oligonucleotides are synthesized directly on the solid support. Methods for synthesizing oligonucleotide arrays are known in the art. See, for example, Fodor et al., U.S. Pat. No. 5,744,305. The sequences of the oligonucleotides represent portions of the sequences of a particular gene to be detected above. Generally, the lengths of oligonucleotides are about 10 to 50 nucleotides (e.g., about 15, 20, 25, 30, 35, 40, or 45 nucleotides).

Tumor Tissue Arrays Tumor tissue arrays can be used to assay protein expression levels or genomic integrity to verify gene expression and CGH information generated from nucleic acid arrays. Information from tumor tissue arrays can be added to a database described herein, and the information can be incorporated into algorithms that will predict appropriate primary and secondary therapeutic agents based on tumor type and molecular phenotype.

To generate a tumor tissue array, tumor and benign control specimens can be obtained and fixed, such as formalin-fixed and paraffin-embedded. Information regarding the tumors, including, but not limited to, stage and clinical information about response to chemotherapy and overall survival can be collected or obtained. More than one sample per tumor specimen can be arrayed. For example, 2, 3, 4, 5, or more samples can be arrayed to account for heterogeneity in the samples. The array can also include a number of normal specimens to serve as controls. In addition, a progression array can be generated which can have the spectrum of pre-malignant lesions of a tumor type, such as a melanoma, which can be accessed to determine relevance to stage of tumor development. For example, a core tissue biopsy specimen, having, for example, a diameter of about 0.6 mm can be taken from the least differentiated regions of individual paraffin-embedded melanomas (donor blocks) and precisely arrayed into a new recipient paraffin block (35-20 mm) with a precision instrument, such as from Beecher Instruments (Silver Spring, Md.) or with a custom made robotic automated arrayer. After the block construction is completed, sections of about 5 mm can be cut with a microtome by use of an adhesive-coated tape sectioning system (Instrumedics, Hackensack, N.J.) to support the adhesion of the array elements. The presence of tumor tissue on the arrayed samples can be verified by a stain, such as a hematoxylin-eosin-stain.

Immunohistochemical analysis (IHC) of a tumor tissue array can be customized and optimized for each antibody. Antigen retrieval can be performed by treatment of the tumor tissue array in a pressure cooker for 5 minutes. Standard indirect immunoperoxidase procedures can be used for immunohistochemistry. A target specific primary antibody and a secondary antibody visualized by, for example, diaminobenzidine as a chromogen. The primary antibodies can be omitted for negative staining controls. The intensity of the cytoplasmic staining can be classified into groups, such as negative, weak, intermediate, and strong staining groups. Alternatively, or in addition, FISH analysis can be performed to validate gene copy number change. A bacterial artificial chromosome (BAC) clone or another large insert clone can be used in addition to or instead of IHC.

IHC and FISH data can be analyzed by statistical methods. For example, contingency table analyses and chi-square tests can be performed to assess the relationship between histological tumor type, grade, stage, and target gene expression/copy number. Survival curves can be plotted according to Kaplan-Meier (Kaplan and Meier et al., J. Am. Stat. Assoc. 53:457-481, 1958). A log rank test can be applied to examine the relationship between grade, stage, or expression/amplification level and tumor recurrence, progression, or survival.

The information gained from a tumor tissue array can be stored in a database, such as a database dedicated to the storage of tumor tissue array data, or any database described herein.

Proteomics

In addition to, or in an alternative to, the genomic approaches discussed above, targets for secondary therapeutic agents can be identified through proteomic methods. Proteomic methods are useful for the identification of proteins in cells and/or tissues. Accordingly, a protein profile of a tumor can be determined before the administration of a primary therapeutic, and again after administration of the primary therapeutic and after tumor progression. Protein microarrays (or protein microchips), are useful for this purpose. As described above for nucleic acid arrays, a protein microarray can include a subset or collection of proteins previously found to be expressed in a tumor cell. Proteins that are determined to be increased or decreased in levels following administration of the primary therapeutic are candidate target proteins for a secondary therapeutic agent. Furthermore, a gene (or the corresponding RNA) encoding a protein that is observed to be increased or decreased in its levels following administration of a primary therapy is a candidate target for a secondary therapy.

A protein microarray suitable for use in the methods described herein can be prepared by a number of methods known in the art. See, for example, methods disclosed in MacBeath and Schreiber (Science 289:1760-1763, 2000), PCT Publication Nos. WO 00/4389A2, WO 00/04382. WO 99/60156, WO 99/39210, WO 00/54046, and WO 99/36576., and U.S. Pat. Nos. 6,087,102, 6,139,831, 6,087,103.

Detection of the proteins can be by the use of peptidic probes, such as antibodies (e.g. polyclonal, monoclonal, and binding fragments thereof); peptides with high affinity to a target protein, as well as analogues and mimetics thereof; ligands, receptors, and the like. Peptidic probes may be obtained from naturally occurring sources or synthesized using available technologies.

Probes can be directly detectable labels including isotopic and fluorescent moieties incorporated into (e.g., covalently bonded to) a moiety of the probe. Isotopic moieties or labels of interest include ³²P, ³³P, ³⁵S, ¹²⁵I, and the like. Fluorescent moieties or labels of interest include coumarin and its derivatives, e.g. 7-amino-4-methylcoumarin, aminocoumarin, bodipy dyes, such as Bodipy FL, cascade blue, fluorescein and its derivatives, e.g. fluorescein isothiocyanate, Oregon green, rhodamine dyes, e.g., Texas red, tetramethylrhodamine, eosins and erythrosins, cyanine dyes, e.g., Cy3 and Cy5, fluorescent energy transfer dyes, such as thiazole orange-ethidium heterodimer, TOTAB, etc. Labels may also be members of a signal producing system that act in concert with one or more additional members of the same system to provide a detectable signal. Illustrative of such labels are members of a specific binding pair, such as ligands, e.g., biotin, fluorescein, digoxigenin, antigen, polyvalent cations, chelator groups and the like, where the members specifically bind to additional members of the signal producing system, where the additional members provide a detectable signal either directly or indirectly, e.g. antibody conjugated to a fluorescent moiety or an enzymatic moiety capable of converting a substrate to a chromogenic product, such as alkaline phosphatase conjugate antibody and the like. Additional labels of interest include those that provide for signal only when the probe with which they are associated is specifically bound to a target molecule, such as “molecular beacons” (see Tyagi & Kramer, Nature Biotechnology 14:303, 1996; and EP 0 070 685 B1). Other useful labels are known in the art.

Therapeutic Methods

The new methods and compositions featured in the invention can be used to determine an appropriate therapy for an individual. For example, a sample of a tumor (e.g., a tissue obtained by a biopsy procedure, such as a needle biopsy) can be provided from the individual, such as before a primary therapy is administered. The gene expression profile of the tumor can be determined, such as by a nucleic acid array (or protein array) technology, and the expression profile can be compared to a database as described herein. Optionally, a CGH analysis can be performed to assay for gene amplification. Also optionally, a software program linked to the database can generate a recommendation for a primary cancer therapy based on the gene expression profile of the tumor (and CGH data, if determined), and other information relating to the human (e.g., age, gender, family history, etc.). In another option, a software program is not used, but a healthcare provider will consult the information stored in the database and will make a decision to administer or prescribe a particular drug based on the comparison of the expression profile of the tumor and information in the database. A healthcare provider can be, for example, a doctor, nurse, or other practitioner.

Following treatment with a primary cancer therapy, the patient will be monitored for an improvement or worsening of the cancer. A tumor tissue sample (such as a biopsy) can be taken at any stage of treatment. In particular, a tumor tissue sample can be taken upon tumor progression, which can be determined by tumor growth or metastasis. A gene expression profile and, optionally, CGH analysis, can be determined, and one or more secondary therapeutic agents can be administered to increase, or restore, the sensitivity of the tumor to the primary therapy.

In one alternative, the database and, optionally, the software described above will make a prediction based on the pre-treatment expression profile, as to which genes will be upregulated upon treatment with the recommended primary therapy. One or more appropriate secondary therapeutics can be selected based on the prediction, and the one or more secondary therapeutics can be administered with the primary therapeutic from the first day of treatment. The patient can further be monitored for an effect on tumor progression.

The invention is illustrated by the following examples, which should not be construed as further limiting.

### EXAMPLES

### Example 1

### Agilent 60-Mer Oligonucleotide Arrays can be Used to Detect Gene Copy Number Changes at the Single-Copy Level

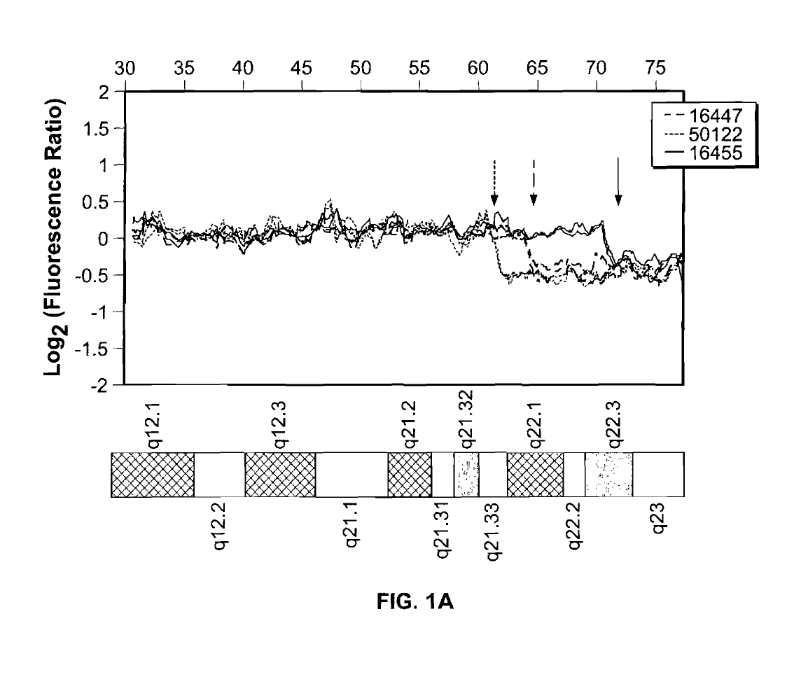

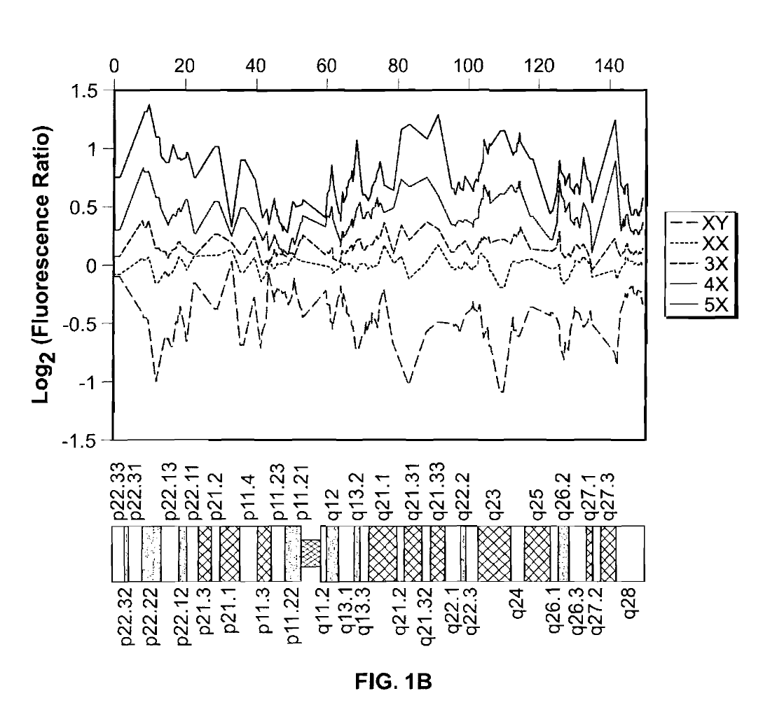

Chromosome 18q and the X chromosome were analyzed by comparative genomic hybridization (CGH). Duplicate hybridizations from three separate 18q-cell lines were hybridized to 60-mer oligonucleotide microarrays from Agilent Technologies (Palo Alto, Calif.). The data is illustrated in FIG. 1A: a 1 MB moving average of the log₂ fluorescence ratios of chromosome 18 oligonucleotide array probes are plotted as a function of chromosomal position. The graph in FIG. 1A illustrates that single copy number changes in gene expression are visible using this approach. FIG. 1B is a similar graph illustrating different copy numbers detected by analysis of the X chromosome. According to FIG. 1B, five copies of the X chromosome (“5X”) generate the highest Log₂ (fluorescence ratio); an XY chromosome pair generates the lowest signal.

### Example 2

### Genes that have Amplified Copy Number will also Often be Overexpressed

CGH and microarray data were combined to compare gene amplification data and expression microarray data from a neuroblastoma cell line. At least three amplicons were identified from chromosome 12, and several overexpressed genes were identified at chromosome position 12q24. CDK4 and MDM2 were two oncogenes identified as being overexpressed and for having an amplified gene copy number. Other amplified and overexpressed candidate genes were also identified.

### Example 3

### Drug Target Validation was Assayed by RNAi

The Cancer Drug Development Laboratory (CDDL) within Translational Genomics Research Institute (TGen) (Phoenix, Ariz.) generated several siRNA libraries and developed tools and rules for generation and high throughput utilization of siRNA. A series of libraries have been used in phenotype screening studies to determine their effects on survival, sensitization to various drugs, and a number of cell based assays and molecular endpoints including CDKN1A promoter activation, apoptosis, and cell cycle profiling.

Transfection protocols for RNAi were optimized for use in 15 different cancer cell lines (including melanoma), both in microtiter well format as well as in more sophisticated platforms such as RNAi microarrays (Mousses et al., Genome Research 13: 2341-7, 2003). Two to three siRNAs were used to test drug-target validation for each amplified candidate.

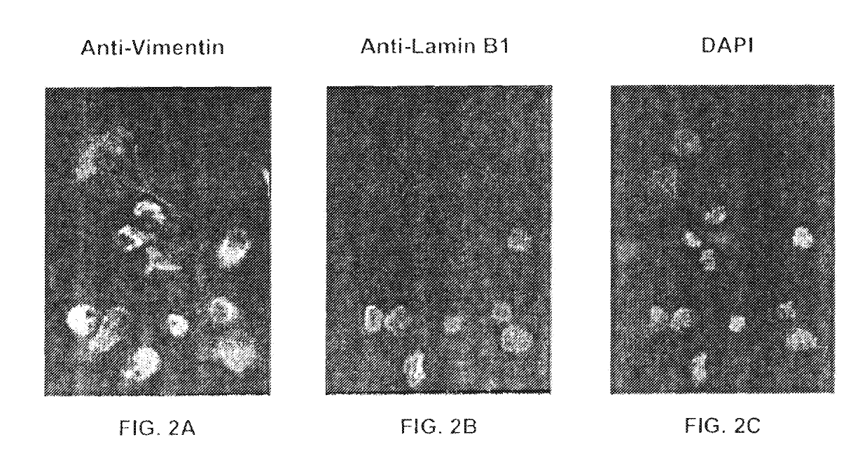

An example of gene silencing in tumor cells using siRNA is shown in FIG. 2 for a pancreas cancer cell line. Panc1 cells were grown to 60% confluency and treated with Lamin B1 siRNA complexed with Lipofectamine 2000. Treated cells were fixed and expression changes were demonstrated using anti-Lamin B1 antibodies. FIG. 2 shows the silencing of Lamin B1 expression with Lamin B1 siRNA. All cells showed nuclear DAPI staining (FIG. 2C) and the expression of vimentin was demonstrated using an anti-vimentin antibody (FIG. 2A). Lamin B1 expression varied but could clearly be seen as silenced in a percentage of cells (FIG. 2B).

Introduction of siRNA into cells was performed by chemical transfection with commercially available cationic lipids. This approach was most amenable to the use of RNAi assays for high-throughput screening (HTS). To develop a highly reproducible and efficient transfection assay, 13 commercially available transfection reagents were screened for their ability to effectively silence GFP in GFP-expressing cell lines. The transfection reagents tested were Lipofectamine 2000, Lipofectin, Oligofectamine and Cellfectin (Invitrogen), siPORT lipid and siPORT amine (Ambion), TransIT-TKO (Mirus), GeneEraser (Stratagene), Ribojuice (Novagen), Jet-SI (Q-biogene), RNAifect (Qiagen), Fugene-6 (Roche) and Exgen-500 (Fermentas).

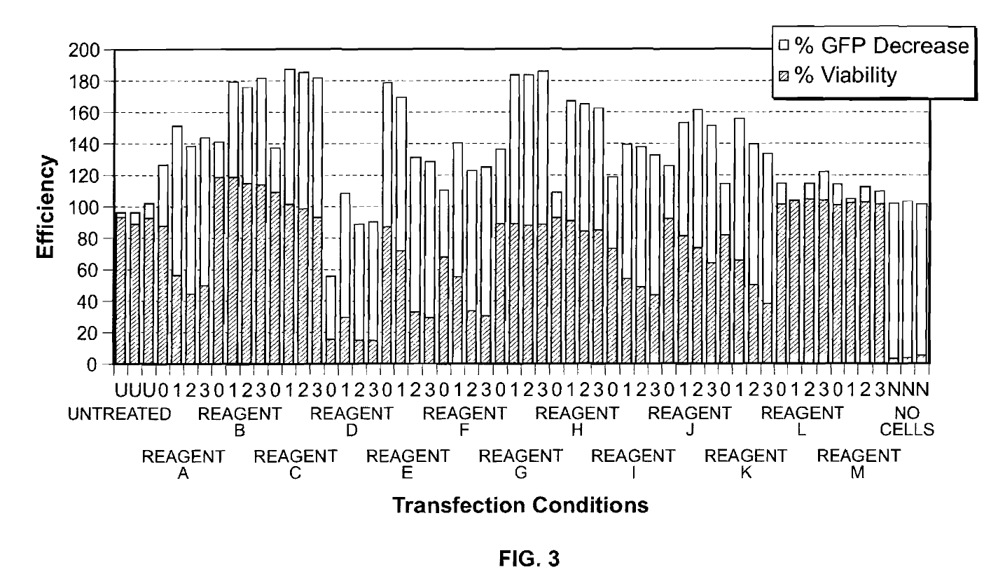

The optimization assay was performed by seeding SK-BR3-EGFP cells into four black clear-bottom 96 well plates (Corning) at a concentration of 5000-7000 cells/well. Cells were incubated 18 hrs prior to transfection. Dilutions of the 13 transfection reagents were prepared in OptiMEM (Invitrogen) for a final complex plate concentration of 0.2 μl/well. The diluted transfection reagent was incubated at room temperature for 20 min. GFP siRNA (Qiagen) was also diluted in OptiMEM to give a final complex plate concentration of 0, 0.1, 0.2, and 0.4 μg/well. The diluted siRNA and diluted transfection reagents were mixed and allowed to complex for 20 min. at room temperature. Growth media was removed from the cells and transfection complex was added. The cells were exposed to the transfection complex for 18 hrs. after which an equal volume of growth media containing 20% FEBS is added, and cells were allowed to grow for another 48 hours. After a total 72 hrs. post-transfection, 20 μl of Cell Titer Blue Reagent (Promega) was added to each well to determine cell viability. The cells were incubated for another four hours. Fluorescence intensity for each plate was obtained using a BMG Polarstar (BMG) with filters for ex 544 nm/em 560 nm. Percent viability values were generated by comparing each RFU from each treatment condition with that of untreated samples. Percent GFP reduction was determined by comparing the difference of (untreated samples-treatment condition) to (untreated samples-no cell controls). The percent viability and percent GFP reduction were added together and plotted to determine efficiency. This method was used to optimize transfection conditions for six different cancer cell lines, and a similar approach was used to examine a series (more than 140) of melanoma cell lines. FIG. 3 shows the efficiency of the 13 transfection reagents in silencing GFP in the SK-BR3 breast cancer cell line.

### Example 4

### Screening Assay Identified 42 siRNAs that Significantly Decrease Cell Survival

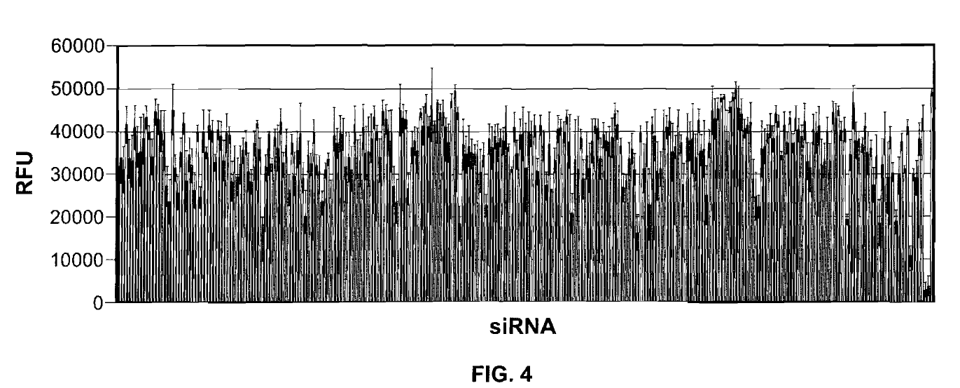

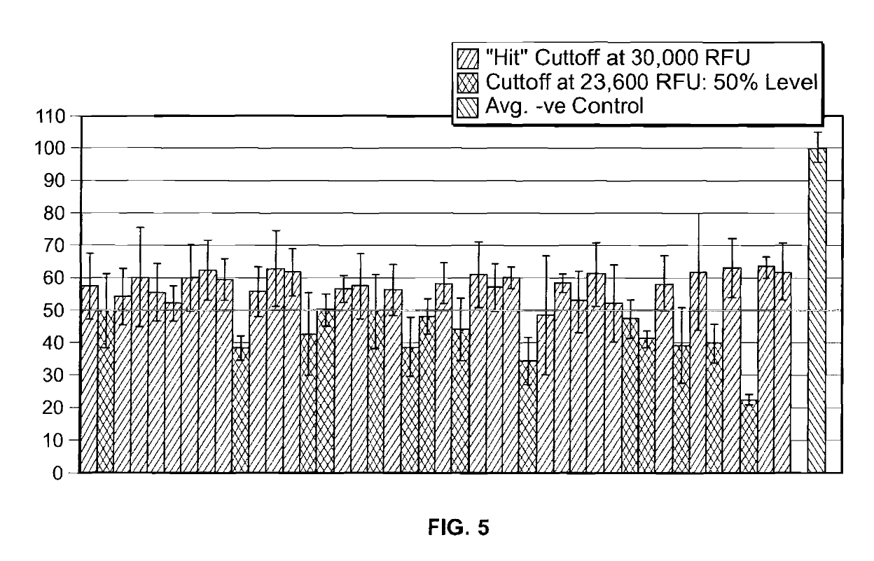

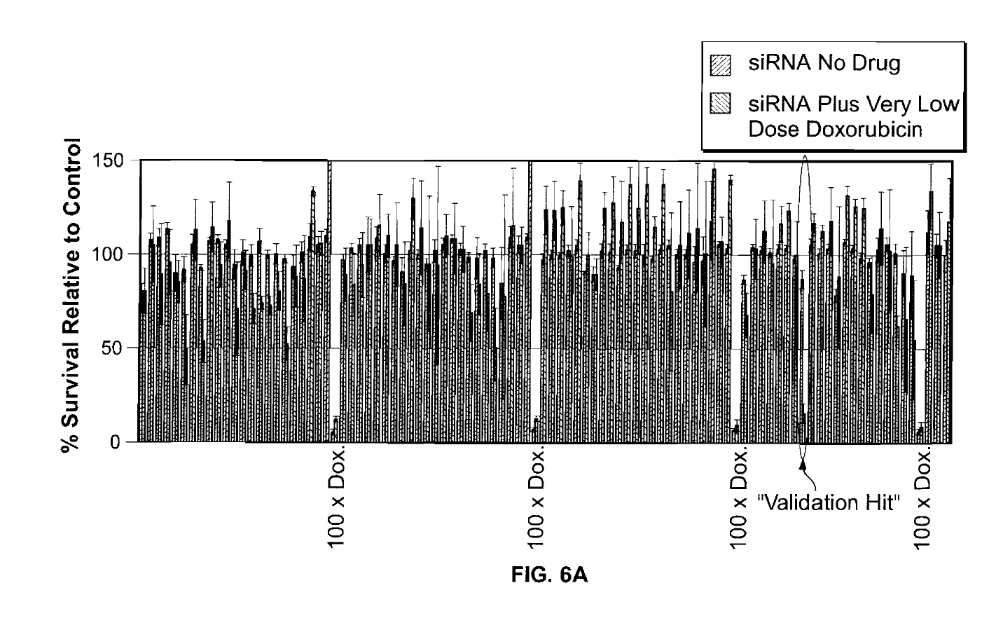

To assay the role of specific cancer-associated genes on cell survival and the reaction of these genes to selected anti-cancer drugs, methods were developed for high throughput RNAi screening of siRNA libraries in which phenotypic changes, such as cell viability were examined. The initial test involved the transfection of HeLa cells with siRNA of 139 cancer-associated genes using Lipofectimine 2000 (Invitrogen) (FIG. 4). HeLa cells were plated onto 14 black, clear-bottom 96-well plates at a concentration of 5000 cells/well. Following an 18 hr. incubation, cells were transfected with 0.2 μg/well of specific siRNA complexed with Lipofectamine 2000 (Invitrogen). In total, 1112 test transfections were performed (4 replicates×2 siRNA×139 cancer genes), and 224 control transfections were performed (4 replicates×56 siRNA control treatments). At 72 hrs. post-transfection, cell viability was determined using Cell Titer Blue Reagent (Promega). Relative Fluorescent Units (RFU) were measured. The average of four replicates was used to plot the effect for each siRNA (FIG. 5), and the standard deviation of the replicates was used to plot error bars. Controls included (i) transfection with no siRNA, (ii) a “no cell” control sample, and (iii) transfection with a scrambled sequence control siRNA (Qiagen). Forty-two siRNAs (15% out of the 278 tested) had a significant decrease in survival, and about 5% of the siRNA from the screened library resulted in a greater than 50% reduction in viability (FIG. 5). The most potent effect was seen with a single siRNA that reduced the number of viable cells down to about 22% of the control.

### Example 5

### Validation of 29 Amplified and Overexpressed Genes for Functional Modulation of Drug Sensitivity

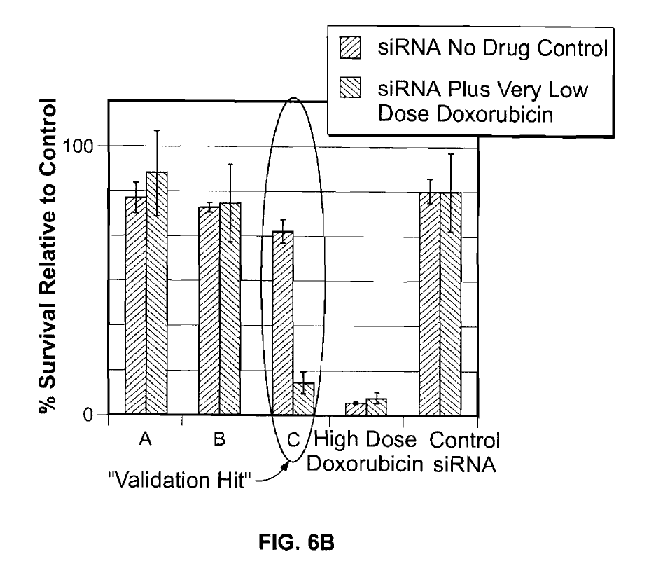

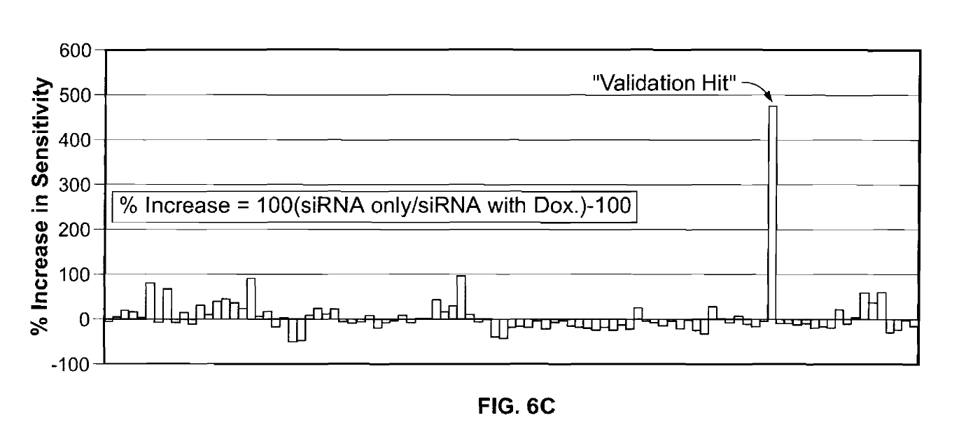

An analysis was conducted by a similar method as described in FIG. 4, with two main differences. First, a different set of siRNAs were used. The siRNAs targeted 29 novel candidate genes that were previously identified by cDNA microarray and CGH analysis to be overexpressed and amplified in 14 breast cancer cell lines (Hyman et al., Cancer Res. 62:6240-5, 2002). There were 76 test siRNAs (2 or 3 siRNA per gene plus 6 control siRNAs). The second difference in this study was that the number of experiments was doubled to include a low dose doxorubicin (“Dox”) treatment. Four replicates of the no-drug control, and four replicates of the low dose drug were prepared for a total of 8 wells treated with each siRNA. The HeLa cells were transfected with siRNA as described above, and the cells were allowed to incubate for 24 hours to achieve silencing of the targeted genes. A low dose of Dox was added to the cells, and they were incubated for an additional 48 hours before assaying for number of surviving cells (the low dose of Dox was empirically determined through a dose response experiment that allowed the selection of a dose that was 50 fold lower than the LD90 and showed no effect on HeLa cell survival). FIG. 6 shows the effects of pretreatment with various siRNA on survival with and without a low non-toxic dose of Dox. FIG. 6A shows paired siRNA pretreatments without drug (light gray bars) and with 0.5 μg/ml Dox (dark gray bars). In most siRNA pretreatment, the effect of low dose Dox on cell survival was not significantly different than the “no drug” controls. There was one striking exception that is enlarged in FIG. 6B: the siRNA “C” in combination with the drug caused a striking decrease in cell survival. The level of sensitization achieved was almost as high as the high dose Doxorubicin control wells where both groups were treated with 40 times the low dose of the drug. FIG. 6C shows the same data from FIG. 6A plotted as a percentage increase in sensitivity relative to the untreated sample. FIG. 6C illustrates that the observed effect of FIG. 6A and FIG. 6B was almost a 500% increase in sensitivity. FIG. 6C also shows that there were about 4 “validation hits” that had about a 100% increase in drug sensitivity.

### OTHER EMBODIMENTS

A number of embodiments of the invention have been described. Nevertheless, it will be understood that various modifications may be made without departing from the spirit and scope of the invention. Accordingly, other embodiments are within the scope of the following claims.

What is claimed is:

1. A method of treating a patient having a tumor, said method comprising:
(a) determining a gene expression profile of said tumor;
(b) comparing said gene expression profile with information in said database of claim 1; and
(c) selecting a primary therapy to treat said tumor based on the information in said database.

2. The method of claim 1, further comprising:
(d) identifying a gene that is modified in response to said primary therapy, and
(c) administering a secondary therapeutic agent to alter activity of said gene, or an RNA or protein expressed from said gene.

3. The method of claim 1, further comprising determining a gene expression profile of said tumor following treatment of said tumor with said primary therapy.

4. The method of claim 2, wherein said secondary therapeutic agent is an siRNA, antisense RNA, antibody, or small molecule inhibitor.

5. A method of selecting a drug profile for a patient having a tumor, the method comprising:
(a) determining a gene expression profile of said tumor;
(b) comparing said gene expression profile to said database of claim 1; and
(c) selecting a drug to treat said tumor based on the information in said database.

6. A method of identifying a therapeutic agent to treat a tumor, the method comprising:
(a) providing a test sample of tumor cells and a control sample of tumor cells;
(b) contacting said test sample with a primary therapy drug and a test secondary therapeutic agent, and contacting said control sample with said primary therapy drug; and
(c) assaying survivability of said test sample and said control sample, wherein a decrease in cell survivability in said test sample as compared to said control sample is an indication that the test secondary therapeutic agent can be used to treat a tumor.

7. A method of monitoring a tumor in a patient comprising:
(a) determining a gene expression profile of said tumor before administration of an anti-tumor therapy;
(b) determining a gene expression profile of said tumor after administration of an anti-tumor therapy; and
(c) comparing said gene expression profiles of (a) and (b) to identify a gene that has modified expression in response to said anti-tumor therapy.

8. A method of treating a patient having a tumor, said method comprising:
(a) determining a gene expression profile of said tumor;
(b) administering a primary therapy to treat said tumor in said patient;
(c) determining a gene expression profile of said tumor after administration of said therapy; and
(d) comparing said gene expression profiles of (a) and (c) to identify a gene that is modified following administration of said anti-tumor therapy.

9. The method of claim 8, further comprising administering a secondary therapeutic agent to alter activity of said gene, or an RNA or protein expressed from said gene.
